(19) United States
(12) Patent Application Publication
(10) Pub. No.: US 2022/0347548 A1
(43) Pub. Date: Nov. 3, 2022

(54) SYSTEMS AND METHODS FOR HAPTIC SIMULATION IN EXERCISE DEVICES

(71) Applicant: iFIT Inc., Logan, UT (US)

(72) Inventor: Scott R. Watterson, Logan, UT (US)

(21) Appl. No.: 17/733,671

(22) Filed: Apr. 29, 2022

Related U.S. Application Data
(60) Provisional application No. 63/182,469, filed on Apr. 30, 2021.

(51) Int. Cl. A63B 71/06; A63B 22/02; A63B 22/06

(52) U.S. Cl. CPC A63B 71/0619; A63B 22/02; A63B 22/0605; A63B 2071/0655; A63B 2071/0638

(57) ABSTRACT

A haptic simulation exercise device includes a frame, a contact point supported by the frame, and an actuator. The contact point is configured to allow user contact with the exercise device during use of the exercise device. The actuator is configured to displace the contact point relative to the frame.

### CROSS-REFERENCE TO RELATED APPLICATIONS

This application claims priority to and the benefit of United States Provisional Patent Application No. 63/182,469, filed on Apr. 30, 2021, which is hereby incorporated by reference in its entirety.

### BACKGROUND

### Background and Relevant Art

Exercise devices simulate many of the aspects of outdoor exercises using a stationary device, which is conventionally used indoors as an alternative to the outdoor exercise. Exercise devices provide a controlled environment with improved safety and less distractions than the outdoor activities simulated by the exercise devices, such as bicycling, running, rowing, or hiking. Exercise devices allow the user to focus on efficiency, comfort, and convenience without the concerns of external factors.

In simulating the outdoor activity, an exercise device may present video and/or audio information of the outdoor activity, such as running on a beach or riding a bicycle on a dirt road, to the user. A disconnect exists, however, between what the user sees and hears in the presented video and audio information and what the user feels through their interaction with the exercise device.

### BRIEF SUMMARY

In some embodiments, a haptic simulation exercise device includes a frame, a contact point supported by the frame, and an actuator. The contact point is configured to allow user contact with the exercise device during use of the exercise device. The actuator is configured to displace the contact point relative to the frame.

This Summary is provided to introduce a selection of concepts in a simplified form that are further described below in the Detailed Description. This Summary is not intended to identify key features or essential features of the claimed subject matter.

Additional features and advantages will be set forth in the description which follows, and in part will be obvious from the description, or may be learned by the practice of the teachings herein. Features and advantages of the disclosure may be realized and obtained by means of the instruments and combinations particularly pointed out in the appended claims. Features of the present disclosure will become more fully apparent from the following description and appended claims or may be learned by the practice of the disclosure as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1 is a perspective view of an exercise bicycle including haptic simulation, according to at least one embodiment of the present disclosure;

FIG. 2 is a perspective view of a treadmill including haptic simulation, according to at least one embodiment of the present disclosure;

FIG. 3 is a user-perspective view of an exercise bicycle including single-axis haptic simulation, according to at least one embodiment of the present disclosure;

FIG. 4-1 is a user-perspective view of an exercise bicycle including multiple-axis vertical haptic simulation, according to at least one embodiment of the present disclosure;

FIG. 4-2 is a user-perspective view of the exercise bicycle of FIG. 4-1 including multiple-axis rotational haptic simulation, according to at least one embodiment of the present disclosure;

FIG. 5 is a side view of an exercise bicycle including haptic simulation though the saddle, according to at least one embodiment of the present disclosure;

FIG. 6 is a side view of an exercise bicycle including haptic simulation through the saddle relative to a tiltable frame, according to at least one embodiment of the present disclosure;

FIG. 7 is perspective view of a treadmill including footstrike detection for haptic simulation, according to at least one embodiment of the present disclosure;

FIG. 8 is top view of a treadmill including a grid of actuators for haptic simulation, according to at least one embodiment of the present disclosure;

FIG. 9 is top view of a treadmill including an array of longitudinal actuators for haptic simulation, according to at least one embodiment of the present disclosure;

FIG. 10 is schematic view of a computing system for providing haptic simulation, according to at least one embodiment of the present disclosure;

FIG. 11 is a flowchart illustrating a method of providing haptic simulation, according to at least one embodiment of the present disclosure;

FIG. 12 is a flowchart illustrating a method of calculating haptic information and providing haptic simulation, according to at least one embodiment of the present disclosure; and

FIG. 13 is schematic representation of a first-person video used to calculate haptic information, according to at least one embodiment of the present disclosure.

### DETAILED DESCRIPTION

The present disclosure relates generally to systems and methods for haptic simulations in an exercise device. More particularly, the exercise devices of the present disclosure include one or more contact points of the exercise device that are movable relative to a frame of the exercise device to increase the user's immersion in a simulated environment.

An exercise device is any mechanical device that is used to provide or replicate a physical activity in a localized space. Exercise devices can include a treadmill, cable or spring resistance machine, weight resistance machine, dumbbells, elliptical machine, stepper machine, stationary bicycle, rowing machine, or any other machine or exercise device. In an example, it should be understood that while a road bicycle may not be an exercise device, as used herein, a bicycle positioned on a stationary trainer device should be considered an exercise device as the bicycle remains in one location while the user rides the bicycle on the stationary trainer device.

In some embodiments, an exercise device includes or is in communication with a display. The display allows a user of the exercise device to view video information as the user engages in exercises. In some embodiments, the display presents video information that simulates a route, path, track, road, trail, or other environment associated with the activity replicated by the exercise device. For example, a display integrated in or in communication with a treadmill may present video information simulating traveling down a road in Oahu, Hawaii at a speed approximately equal to the speed at which the tread belt is moving on the treadmill. The resulting experience for the user is a simulated run down the road presented on the display. Similarly, in another example, a display integrated in or in communication with a stationary bicycle may present video information simulating traveling down a mountain trail in Sedona, Arizona at a speed approximately equal to the speed at which the user moves the pedals of the stationary bicycle. The resulting experience for the user is a simulated mountain bike ride down the trail presented on the display. In yet another example, a display integrated in or in communication with a rowing machine may present video information simulating rowing down the Charles River in Cambridge, Massachusetts at a speed approximately equal to the speed at which the user pulls the handle of the rowing machine. The resulting experience for the user is a simulated row down the river presented on the display.

The exercise device simulates the experience based on simulation data that includes video information as described above. In some embodiments, the simulation data includes audio information. For example, the exercise device may offer one or more simulations such as simulate racing in a stage of a bike race or running away from a dinosaur. In such examples, audio information can increase the immersion of a bike race simulation by simulating cheering fans or sound of another racer approaching from behind on a climb. In another example, audio information can increase the immersion of a dinosaur chase by simulating the roar of the dinosaur behind the runner.

In some embodiments, the simulation data includes haptic information. In the previous examples, the exercise device offers simulations that simulate racing in a stage of a bike race or running away from a dinosaur. In such examples, haptic information can increase the immersion of a bike race simulation by simulating rapidly translating and/or rotating the handlebars to simulate the cobblestone road surface of the Paris-Roubaix bicycle race. In another example, haptic information can increase the immersion of the dinosaur chase by translating the tread belt of the treadmill vertically to simulate the ground shaking with the dinosaur footsteps.

The haptic information may be predetermined and stored on a hardware storage device with the video and/or audio information of the simulation data. In some embodiments, the haptic information may be calculated by the exercise device, a client device, or a workout server based on the video information. For example, a recorded video of a mountain bike ride from a rider's viewpoint (such as video information recorded by a GoPro or other action camera) may include the bicycle handlebars within the frame of the video information. In some embodiments, the system detects the location of the handlebars in the video information and determines the movement of the handlebars relative to the trail surface. The movement of the handlebars may be presented to the exercise device as haptic information and allow the exercise device to simulate the movement of the handlebars in the video information by moving the handlebars of the stationary bicycle relative to a frame of the stationary bicycle.

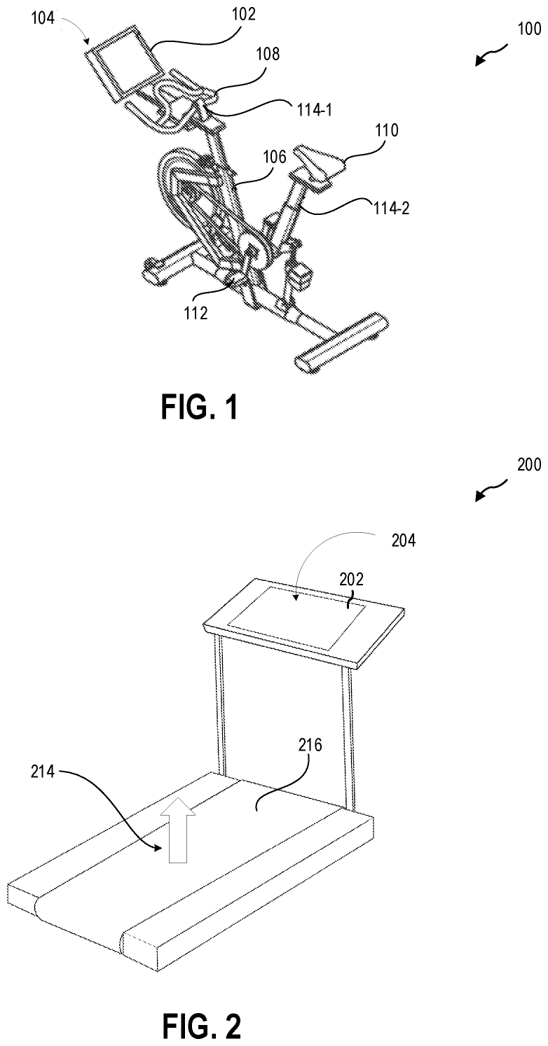

FIG. 1 is a perspective view of an exercise device 100, according to some embodiments of the present disclosure. The embodiment of an exercise device 100 in FIG. 1 is a stationary bicycle with a display 102 integrated into the exercise device 100. In some embodiments, the display 102 may be independent from, but in data communication with, the exercise device 100 and receive video information from a computing device 104 of the exercise device 100. The computing device 104 includes a processor and a hardware storage device with instructions stored thereon that, when executed by the processor, cause the exercise device to perform any of the methods described herein.

In some embodiments, the hardware storage device is any non-transient computer readable medium that may store instructions thereon. The hardware storage device may be any type of solid-state memory; volatile memory, such as static random access memory (SRAM) or dynamic random access memory (DRAM); or non-volatile memory, such as read-only memory (ROM) including programmable ROM (PROM), erasable PROM (ERPOM) or EEPROM; magnetic storage media, such as magnetic tape; platen-based storage device, such as hard disk drives; optical media, such as compact discs (CD), digital video discs (DVD), Blu-ray Discs, or other optical media; removable media such as USB drives; non-removable media such as internal SATA or non-volatile memory express (NVMe) style NAND flash memory, or any other non-transient storage media. In some embodiments, the hardware storage device is local to and/or integrated with the computing device. In some embodiments, the hardware storage device is accessed by the computing device through a network connection.

The exercise device 100 includes one or more contact points supported by a frame 106 with which the user touches, contacts, or engages with the exercise device 100 during the exercise. It should be understood that while the user may touch or contact one or more controls of the exercise device 100, such as a touchscreen of the display 102 or other input devices to provide inputs to the computing device 104 (e.g., volume controls, resistance levels, power buttons), the controls or input devices are not considered the contact points of the exercise device 100 for the purposes of the exercise performed on the exercise device 100. In the embodiment illustrated in FIG. 1, the exercise device 100 is a stationary bicycle and the intended exercise of the stationary bicycle is cycling, therefor, the components of the stationary bicycle used for cycling are considered to be the contact points of the exercise device 100. The contact points of the exercise device 100 include the handlebars 108, the saddle 110, and the pedals 112.

The contact points of the exercise device 100 are movable relative to the frame 106 by one or more actuators 114-1, 114-2 that are in data communication with the computing device 104. A first actuator 114-1 is positioned between the frame 106 and the handlebars 108 to support the handlebars 108 and move the handlebars 108 relative to the frame 106 in response to simulation data processed by the processor of the computing device 104.

Each of the actuators 114-1, 114-2 can include one or more individual actuatable devices, such as including a pair of actuatable devices to allow movement of the contact point in plurality of directions or axes relative to the frame 106. In some embodiments, the actuators 114-1, 114-2 are electromagnetic actuators that use permanent magnets, electromagnets, or combinations thereof to apply magnetic force to move the contact point relative to the frame 106. In some embodiments, the actuators 114-1, 114-2 are fluid piston actuators that use compressible or incompressible fluid in a cylinder to apply a force to a piston that is slidable relative to the cylinder. The fluid may be a gas (e.g., pneumatic piston and cylinder) or a liquid (e.g., hydraulic piston and cylinder) that moves piston relative to the cylinder to move the contact point relative to the frame 106. In some embodiments, the actuators 114-1, 114-2 are mechanical actuators that use gears, springs, elastic or biasing elements, or combinations thereof to apply a compression and/or tension force to move the contact point relative to the frame 106.

In different embodiments, the actuators 114-1, 114-2 move the contact points in different path directions and/or shapes. For example, the actuators 114-1, 114-2 can move the contact point(s) in a linear path. As shown in FIG. 1, the first actuator 114-1 connected to the handlebars 108 may move the handlebars 108 in a linear path up and down to simulate rough surfaces. The second actuator 114-2 connected to the saddle 110 moves the saddle 110 in a linear path up and down to simulate rough surfaces.

In some examples, the actuators 114-1, 114-2 can move the contact point(s) in an arcuate path. As shown in FIG. 1, the first actuator 114-1 connected to the handlebars 108 may move the handlebars 108 in an arcuate path up and toward the user or down and away to simulate rough surfaces. The second actuator 114-2 connected to the saddle 110 may move the saddle 110 in an arcuate path up and toward the user or down and away to simulate rough surfaces. In some embodiments, the travel paths and/or directions can be different between contact points. For example, the handlebars 108 may have an arcuate path and the saddle 110 may have a linear path.

In some examples, an actuator 114-1, 114-2 moves the contact point around a rotational axis. For example, the first actuator 114-1 may rotate the handlebars 108 around a longitudinal axis to simulate the handlebars 108 tilting as would be expected during riding over uneven terrain and/or under heavy pedal loads. The different travel paths or rotational axes may be combined through one or more actuators for a contact point to create a more immersive simulation. In at least one example, the first actuator 114-1 that moves the handlebars 108 may allow for both rotational movement and linear movement, such as lifting the handlebars while tilting to the user's left.

The actuators 114-1, 114-2 move in response to the simulation data provided by the computing device 104. The simulation data includes haptic information that instructs one or more actuators 114-1, 114-2 to move a contact point coordinated with audio and/or video information of the simulation data that is presented to the user on the display 102 or other audiovisual devices (e.g., speakers or headphones). The haptic information may include instructions for one or more actuators 114-1, 114-2 to move the contact point(s) a nominal or relative (to a total range of motion) amount, at a particular speed, for a particular duration, or combination thereof.

For example, the haptic information may instruct the first actuator 114-1 of the handlebars 108 to move the handlebars 108 in coordination with a tree stump presented on the display 102. The haptic information may instruct the actuator 114-1 at a 3:26 timestamp to translate the handlebars upward by 35 millimeters (mm) at a rate of 100 millimeters per second (mm/s) and return the handlebars 108 to the original position at a rate of 50 mm/s. The higher rate of upward travel may replicate the compression of the fork of the bicycle impacting the tree stump in the video information, while the lower rate of downward travel may replicate the freefall of the handlebars 108 dropping off the top of the tree stump.

In another example, the haptic information provides different instructions to the different actuators. For example, the haptic information may instruct the first actuator 114-1 to move the handlebars 108 by 40 mm while the haptic information instructs the second actuator 114-2 to move the saddle 110 by 25 mm to simulate different wheel paths or different suspension responses to the objects in the displayed video information.

FIG. 2 is a perspective view of another exercise device 200 with haptic simulation integrated into a contact point. The treadmill of FIG. 2 includes at least one actuator 214 for a haptic simulation underneath a tread belt 216. The actuator 214 is in communication with a computing device 204 of the exercise device 200 to receive instructions to move at least a portion of the tread belt 216 in coordination with audio and/or video information presented on a display 202.

As described in relation to FIG. 1, the actuator(s) 214 may be an electromagnetic actuator, a fluid piston actuator, a mechanical actuator, or combinations thereof. In some embodiments, the tread belt 216 is a flexible and/or elastic material and a plurality of actuators 214 move different portions of the tread belt 216 to create contours in the surface of the tread belt 216 to simulate a surface presented on the display 202. For example, a plurality of actuators 214 may move the surface of the tread belt 216 by different amounts in different places to simulate running on a presented beach surface with rippled sand. In another example, the plurality of actuators 214 may move the surface of the tread belt 216 by different amounts in different places to simulate trail running with rocks or roots in the presented trail surface. In another example, the plurality of actuators 214 may move the surface of the tread belt 216 by different amounts in different places to simulate urban running with curbs or potholes in a presented road.

In some embodiments, the entire surface of the tread belt 216 may be movable by the actuator(s) 214 to simulate a global effect in the presented simulation. For example, the entire surface of the tread belt 216 may displace upward before returning to the original position to simulate an explosion in the video information presented on the display 202. In at least one example, the exercise device 200 may simulate an action scene and prompt the user of the exercise device 200 to run to safety by evading objects and/or clearing obstacles in the presented video and/or audio information. The action scene may simulate escaping a boobytrapped temple where the user must run along uneven pathways, crumbling walls, and escape a large boulder following the user. The actuator(s) 214 may move portions or all of the tread belt 216 to simulate the debris from the walls on the running surface and simulate the shaking of the ground as the boulder rolls behind the user.

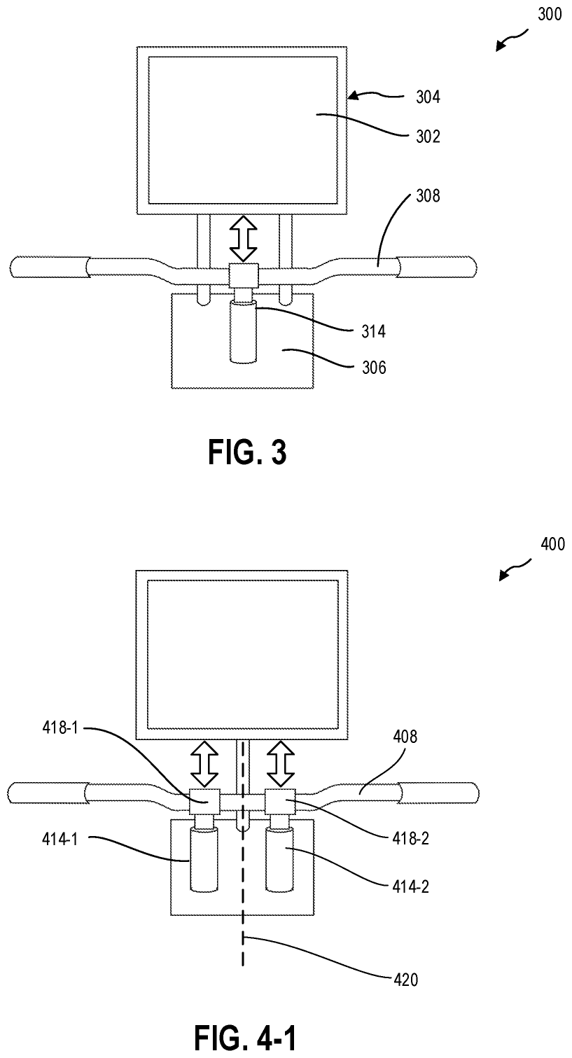

FIG. 3 is a perspective view of another embodiment of an exercise device 300 with handlebars 308 that are movable relative to a frame 306. In some embodiments, such as that illustrated in FIG. 3, the handlebars 308 or other contact point are movable relative to the frame 306 by a single-axis actuator 314. The single-axis actuator 314 may move the handlebars 308 relative to the frame 306 in response to haptic information included in the simulation data.

In some embodiments, the single-axis actuator 314 is in data communication with the computing device 304 of the exercise device 300 to receive instructions including when to actuate, how much to move, how much force to apply, how fast to move, or combinations thereof. For example, a simulation of riding along a rocky trail may include rapid movements of the handlebars 308. In another example, a simulation of an undulating singletrack or bike path may include more gradual movements of the handlebars 308 along the same axis.

The range of motion of the actuator and a range of motion of the contact point movable by the actuator may be different. For example, the actuator may be connected to the contact point (e.g., the actuator 314 that moves the handlebars 308) by being coupled to a linkage or lever that moves the contact point. In other examples, the range of motion of the actuator and a range of motion of the contact point movable by the actuator may be equal, as the actuator and the contact point are directly coupled to one another.

In some embodiments, the range of motion of the contact point is in a range having an upper value, a lower value, or upper and lower values including any of 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, or any values therebetween. In some examples, the range of motion of the contact point is greater than 5 mm. In other examples, the range of motion of the contact point is less than 100 mm. In yet other examples, the range of motion of the contact point is between 5 mm and 100 mm. In yet other examples, the range of motion of the contact point is between 10 mm and 80 mm. In yet other examples, the range of motion of the contact point is about 50 mm.

In some embodiments, the maximum rate of motion of the contact point is in a range having an upper value, a lower value, or upper and lower values including any of 50 mm/s, 100 mm/s, 150 mm/s, 200 mm/s, 250 mm/s, 300 mm/s, 350 mm/s, 400 mm/s, 450 mm/s, 500 mm/s, 550 mm/s, 600 mm/s, 650 mm/s, 700 mm/s, 750 mm/s, 800 mm/s, 850 mm/s, 900 mm/s, 950 mm/s, 1000 mm/s, or any values therebetween. In some examples, the maximum rate of motion of the contact point is greater than 50 mm/s. In other examples, the maximum rate of motion of the contact point is less than 1000 mm/s. In yet other examples, the maximum rate of motion of the contact point is between 50 mm/2 and 1000 mm/s. In yet other examples, the maximum rate of motion of the contact point is between 100 mm/s and 750 mm/s. In yet other examples, the maximum rate of motion of the contact point is greater than 500 mm/s.

FIG. 4-1 and FIG. 4-2 illustrate another embodiment of an exercise device 400 that includes a handlebar 408 (such as on an exercise bicycle as described in relation to FIG. 1) with a plurality of actuators 414-1, 414-2 that provide multi-axis movement of the handlebars 408. In the illustrated embodiment, the actuators 414-1, 414-2 are each directly coupled to the handlebars 408 at rotational couplings 418-1, 418-2, respectively.

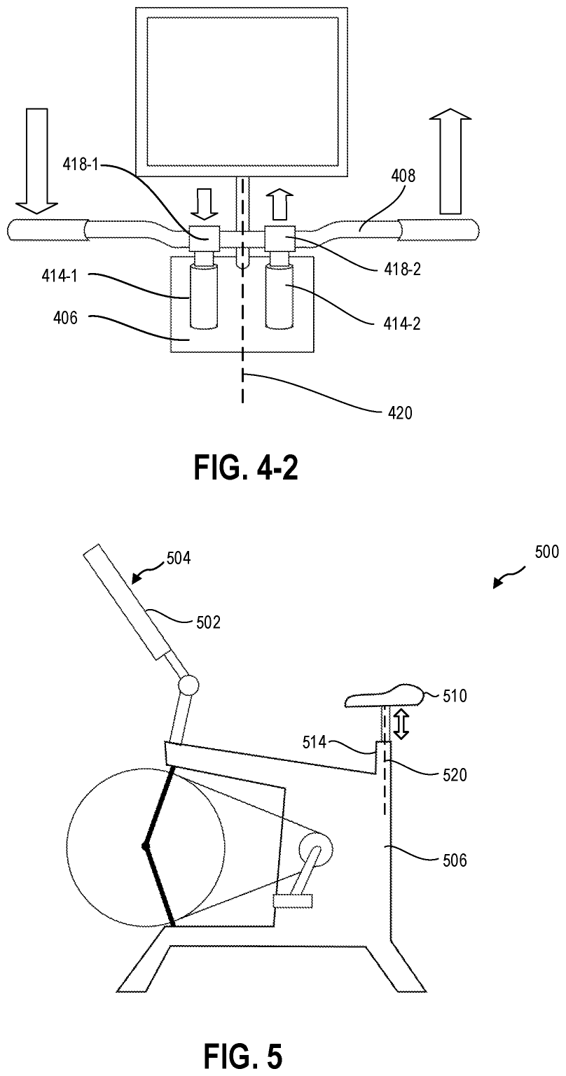

Referring to FIG. 4-1, the first actuator 414-1 and second actuator 414-2 may actuate and/or move the handlebar 408 approximately the same, (e.g., same amount of motion, same rate of motion) such that the handlebars 408 travel along an axis 420 parallel to the axis of the actuators 414-1, 414-2. In such an example, the rotational couplings 418-1, 418-2 may remain at the same angle relative to the handlebars 408.

FIG. 4-2 illustrates an example of rotating the handlebars 408 relative to the frame 406 by actuating the first actuator 414-1 and the second actuator 414-2 differently. For example, the first actuator 414-1 may move a different amount or at different velocity (magnitude or direction) than the second actuator 414-2. The rotational couplings 418-1, 418-2 may allow the handlebars 408 to rotate around an axis perpendicular to the axis 420 of motion of the actuators 414-1, 414-2. In some embodiments, the first actuator 414-1 and the second actuator 414-2 may move in the same direction but different amounts to simulate both a displacement and a rotation of the handlebars 408 relative to the frame 406.

FIG. 5 is a side view of another embodiment of an exercise device 500 with haptic simulation. The exercise device 500 includes an actuator 514 configured to move a saddle 510 relative to a frame 506 of the exercise device 500. The actuator 514 may move the saddle 510 according to haptic information provided by the computing device 504.

In some embodiments, the saddle 510 is movable in relation to the frame 506 to simulate movement associated with audio and/or video information presented to the user via a display 502 or other device. The actuator 514 may move the saddle 510 in an axis 520, as illustrated in FIG. 5. In some embodiments, the saddle 510 may be connected to a plurality of actuators to move the saddle 510 in a different path or around a rotational axis to simulate other types of movement according to the haptic information.

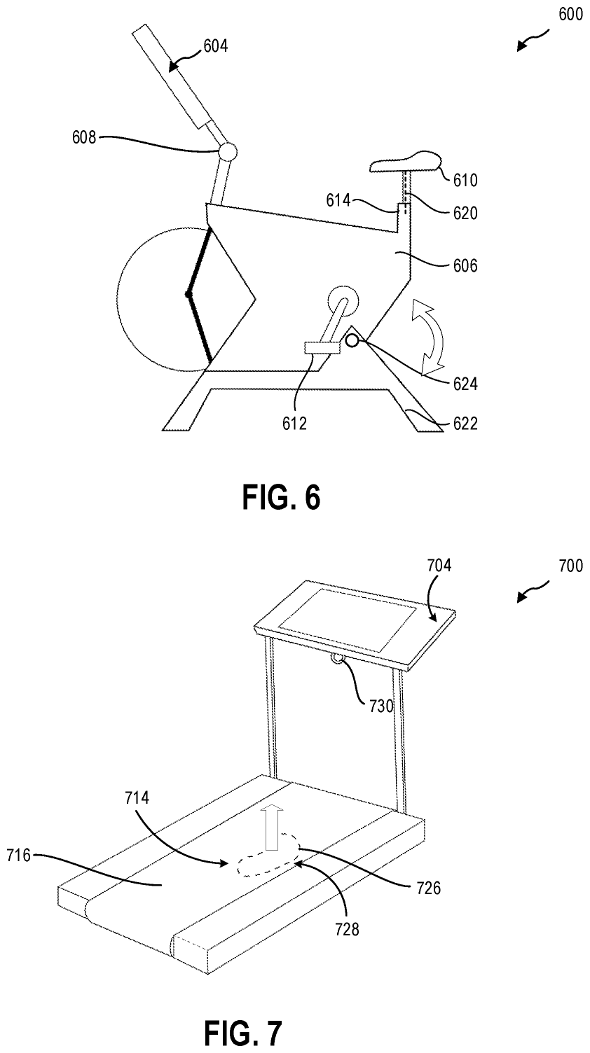

FIG. 6 is a side view of another embodiment of an exercise device 600 with haptic simulation. The exercise device 600 includes an actuator 614 configured to move a saddle 610 relative to a frame 606 of the exercise device 600. The actuator 614 may move the saddle 610 according to haptic information provided by a computing device 604. In some embodiments, the frame 606 is movable (e.g., tiltable) relative to a base 622 that contacts the ground. The frame 606 may connect to the base 622 at a rotational pivot 624 that allows the frame 606 supporting the saddle 610, handlebars 608, and pedals 612 to move relative to the ground. In some examples, the frame 606 tilts around a pivot 624 allowing the exercise device 600 to simulate the angle of climbing up a road, while the haptic simulation of the saddle 610 along an axis 620 allows the exercise device 600 to simulate the surface condition of the road. The computing device 604 may be in data communication with the actuator(s) 614 and a motor to tilt the frame 606 to provide a more immersive simulation.

While the movable frame 606 relative to the base 622 is described herein in relation to an exercise bicycle, it should be understood that a tiltable or movable frame with haptic simulation, where the frame is movable relative to a base, is applicable to other types of exercise devices, such as treadmills, rowing machines, and other devices.

FIG. 7 is a perspective view of another embodiment of an exercise device 700 with haptic simulation. While a user may maintain substantially continuous contact with the contact points of an exercise bicycle, a user will continuously lift, move, and lower their feet relative to the contact point (i.e., tread belt 716) of a treadmill. In some embodiments, an exercise device 700 according to the present disclosure may use contact detection and/or prediction to determine when and/or where to move the contact point(s). In some embodiments, the exercise device 700 may adapt the user's position on the exercise device by moving the contact point at or near the location of a footstrike 726. In some embodiments, the exercise device 700 may conserve energy by only moving the contact point at or near the location of a footstrike 726.

In some embodiments, the exercise device 700 detects the location and time of the footstrike 726. For example, the exercise device 700 may include one or more pressure sensors 728 positioned in or underneath the tread belt 716 to measure an application of force to the tread belt 716. A computing device 704 in communication with the pressure sensor(s) 728 may receive the measurements from the pressure sensor(s) 728 and determine the user's foot has contacted the tread belt 716 at the location of the pressure sensor(s) 728. In some embodiments, a minimum force or pressure measurement may be required for the pressure sensor 728 to transmit the measurement or for the computing device 704 to interpret the measurement as a footstrike 726. The computing device 704 may then send instructions to the actuator(s) 714 associated with the location of the footstrike 726 to move the tread belt 716 and provide haptic simulation in coordination with audio and/or video information.

In some embodiments, the exercise device 700 includes one or more cameras 730 positioned and oriented to monitor the movement and location of the user's feet relative to the tread belt 716. The camera 730 may transmit video data to the computing device 704 to allow the computing device 704 to identify the location of the user's foot when the foot make contact with the tread belt 716 and determine the location of the footstrike 726. The computing device 704 may then send instructions to the actuator(s) 714 associated with the location of the footstrike 726 to move the tread belt 716 and provide haptic simulation in coordination with audio and/or video information.

In some embodiments, the exercise device 700 predicts the location and time of the footstrike 726. For example, the exercise device 700 may include one or more pressure sensors 728 positioned in or underneath the tread belt 716 to measure an application of force to the tread belt 716. The computing device 704 in communication with the pressure sensor(s) 728 may receive the measurements from the pressure sensor(s) 728 and determine the user's foot has contacted the tread belt 716 at the location of the pressure sensor(s) 728. In some embodiments, the computing device 704 may track and average the location and intervals between a sequence of footstrikes 726. The computing device 704 may continue to calculate a rolling average of the recent cadence and/or location of the footstrikes 726 to predict the location of a next footstrike 726. The computing device 704 may then send instructions to the actuator(s) 714 associated with the location of the predicted footstrike 726 to move the tread belt 716 and provide haptic simulation in coordination with audio and/or video information.

In some embodiments, the exercise device 700 includes one or more cameras 730 positioned and oriented to monitor the movement and location of the user's feet relative to the tread belt 716. The camera 730 may transmit video data to the computing device 704 to allow the computing device 704 to identify the location of the user's foot when the foot make contact with the tread belt 716 and determine the location of the footstrike 726. In some embodiments, the computing device 704 may track and average the location and intervals between a sequence of footstrikes 726. The computing device 704 may continue to calculate a rolling average of the recent cadence and/or location of the footstrikes 726 to predict the location of a next footstrike 726. The computing device 704 may then send instructions to the actuator(s) 714 associated with the location of the predicted footstrike 726 to move the tread belt 716 and provide haptic simulation in coordination with audio and/or video information.

In some embodiments, the camera 730 may measure the movement of the user's foot and transmit the location and movement of the user's foot to the computing device 704. The computing device 704 may track the motion of the user's foot immediately prior to the user's foot contacting the tread belt 716 and predict the location of the footstrike 726. The computing device 704 may then send instructions to the actuator(s) 714 associated with the location of the predicted footstrike 726 to move the tread belt 716 and provide haptic simulation in coordination with audio and/or video information.

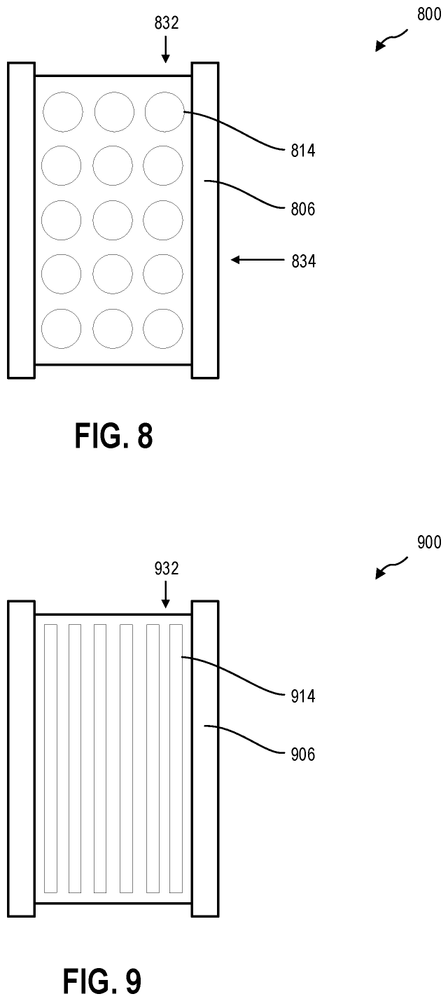

To provide haptic simulation of a running surface, the exercise device 700 may include a plurality of actuators 714 to move the tread belt 716 perpendicular to the conventional movement of the rotating tread belt 716. FIG. 8 is a top view of another embodiment of an exercise device 800 according to the present disclosure. The exercise device 800 includes a frame 806 that supports a plurality of actuators 814. As described herein, the actuators 814 may include an electromagnetic actuator, a fluid piston actuator, a mechanical actuator, or combinations thereof.

In some embodiments, the exercise device 800 includes a grid of actuators 814, such as shown in FIG. 8. The grid may position the plurality of actuators 814 in a plurality of columns 832 and rows 834. In some embodiments, the grid is a 3x5 grid, as illustrated in FIG. 8. In some embodiments, the grid has more or less than 3 columns 832, and in some embodiments, the grid has more or less than 5 rows 834.

Because the tread belt (not shown in FIG. 8) moves relative to the frame 806 and the actuators 814, the location of the user's foot will move in relation to the actuators 814 while in contact with the tread belt. In some embodiments, a plurality of actuators 814 in a column 832 moves simultaneously to simulate the user moving relative to the running surface. In some embodiments, the plurality of actuators 814 in a column 832 moves sequentially based on the speed of the tread belt.

FIG. 9 is a top view of another embodiment of an exercise device 900 with a plurality of elongated actuators 914. The exercise device 900 includes actuators 914 positioned in a plurality of columns 932, where each column 932 includes one actuator that is substantially the full length of the frame 906. As the user's foot does not move laterally relative to the direction of the tread belt rotation while the user's foot is in contact with the tread belt, the entire column 932 may be actuated to simulate the user moving relative to the running surface.

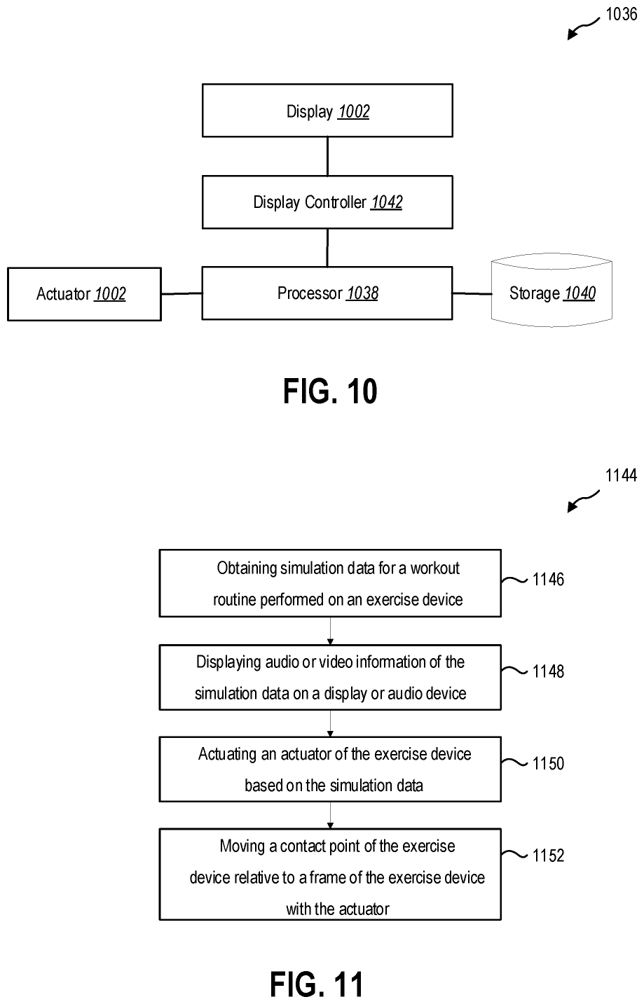

An exercise device according to the present disclosure may include a plurality of electronic components in data communication. FIG. 10 is a schematic representation of an electronic system 1036 of any exercise device described herein. The electronic system 1036 includes a processor 1038 and a hardware storage device 1040. In some embodiments, the hardware storage device 1040 is any non-transient computer readable medium that may store instructions thereon. The hardware storage device 1040 may be any type of solid-state memory; volatile memory, such as static random access memory (SRAM) or dynamic random access memory (DRAM); or non-volatile memory, such as read-only memory (ROM) including programmable ROM (PROM), erasable PROM (ERPOM) or EEPROM; magnetic storage media, such as magnetic tape; platen-based storage device, such as hard disk drives; optical media, such as compact discs (CD), digital video discs (DVD), Blu-ray Discs, or other optical media; removable media such as USB drives; non-removable media such as internal SATA or non-volatile memory express (NVMe) style NAND flash memory, or any other non-transient storage media. In some embodiments, the hardware storage device 1040 is local to and/or integrated with the processor 1038. In some embodiments, the hardware storage device 1040 is accessed by the processor 1038 through a network connection.

The processor 1038 is further in communication with a display controller 1042. The processor 1038 receives simulation data from the hardware storage device 1040. In some embodiments, the simulation data includes video information and haptic information. The processor 1038 transmits video information to the display controller 1042 and instructions based on the haptic information to the actuator 1014. The display controller 1042 communicates with the display 1002 to present the video information to a user, while the actuator 1014 moves at least one contact point of the exercise device to provide haptic simulation to the user. The processor 1038 may coordinate the presentation of the video information and haptic information to the user such that the user perceives the exercise device moving in coordination with the displayed video. In some embodiments, the processor 1038 is further in communication with the one or more sensors and/or cameras to monitor the position and/or movement of the user. The processor 1038 may transmit instructions or one or more actuators of a plurality of actuators based on the measurements from the sensors and/or cameras.

FIG. 11 is a flowchart illustrating a method 1144 of providing haptic simulation in an exercise device, according to some embodiments of the present disclosure. The method 1144 includes obtaining simulation data for a workout routine performed on an exercise device at 1146. In some embodiments, the simulation data is obtained from local hardware storage device. In some embodiments, the simulation data is obtained from a remote hardware storage device, such as by downloading or streaming the simulation data from a remote server or datacenter. A workout routine is a predetermined set of instructions that may be provided to a user. Following the workout routine can guide, entertain, or encourage the user through one or more exercises to produce workout information. The workout routine may include haptic information, video information, audio information, text information, still images, or combinations thereof to communicate the workout routine to the user.

The method 1144 further includes displaying video information of the simulation data on a display (such as display 102 described in relation to FIG. 1) at 1148. In some embodiments, the method 1144 further includes playing audio information of the simulation data at 1148. In some embodiments, the simulation data includes both audio information and video information. The audio and/or video information provides visual and/or auditory immersion to which the haptic simulation(s) add further immersion.

The haptic information of the simulation data is used in actuating an actuator of the exercise device at 1150, and the method 1144 includes moving a contact point of the exercise device relative to the frame of the exercise device with the actuator at 1152. In some embodiments, the frame is further movable relative to the base to change an inclination or declination of the exercise device.

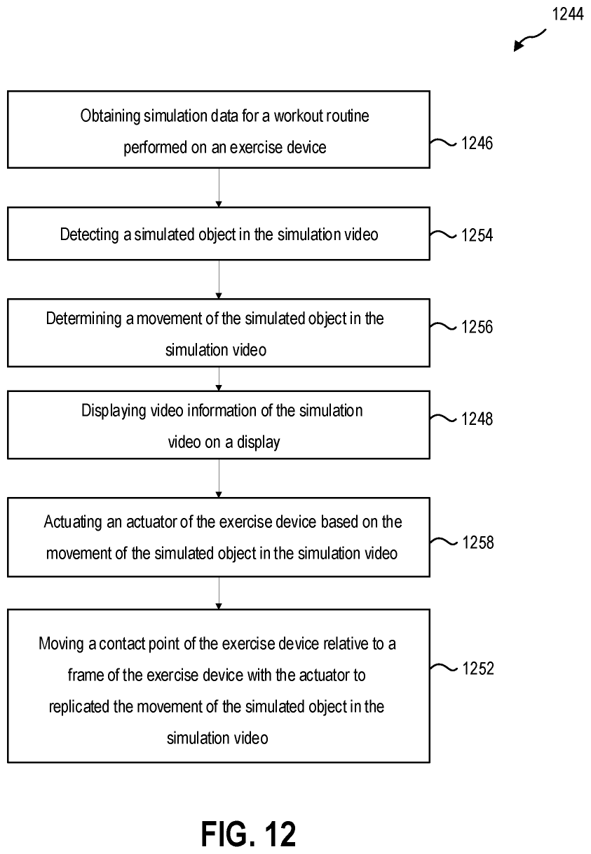

While the method 1144 includes actuating an actuator of the exercise device based on the simulation data, in some embodiments, the simulation data does not include haptic information, and the processor may calculate haptic information from the simulation data as described in relation to FIG. 12.

FIG. 12 is a flowchart illustrating another embodiment of a method 1244 of providing haptic simulation in an exercise device. The method 1244 includes obtaining simulation data for a workout routine performed on an exercise device at 1246, similar to or the same as described in relation to FIG. 11. In some embodiments, the simulation data is obtained from local hardware storage device. In some embodiments, the simulation data is obtained from a remote hardware storage device, such as by downloading or streaming the simulation data from a remote server or datacenter.

The method 1244 may further include detecting a simulated object in the simulation video or video information at 1254 and determining a movement of the simulated object in simulation video or video information at 1256. In some embodiments, the simulated object is an object in the simulation video or video information that is analogous to a contact point of the exercise device. For example, the simulation video or video information may be a first-person perspective of a rider riding a mountain bike of a trail. The simulation video or video information may include the handlebars in the frame of the simulation video or video information, and a computing device or processor may detect the presence of the handlebars. In other examples, the simulation video or video information may include a running surface in the frame of the simulation video or video information, allowing the system to detected curbs, rocks, or roots in the video and simulate the running surface with actuators that move the tread belt.

The method 1244 further includes displaying video information of the simulation data on a display (such as display 102 described in relation to FIG. 1) at 1248. In some embodiments, the method 1244 further includes playing audio information of the simulation data at 1248. In some embodiments, the simulation data includes both audio information and video information. The audio and/or video information provides visual and/or auditory immersion to which the haptic simulation(s) add further immersion.

In some embodiments, the simulated object in the simulation video or video information is detected relative to a horizon line or other reference point to measure the amount of displacement of the simulated object in the video. In some examples, the simulated object is measured relative to other stationary objects in the environment to measure the amount of movement. In some embodiments, the movement of the simulated object is determined by a machine learning (ML) that refines through iterations.

A machine learning model (ML model) according to the present disclosure refers to a computer algorithm or model (e.g., a classification model, a regression model, a language model, an object detection model) that can be tuned (e.g., trained) based on training input to approximate unknown functions. For example, a machine learning model may refer to a neural network or other machine learning algorithm or architecture that learns and approximates complex functions and generate outputs based on a plurality of inputs provided to the machine learning model. In some embodiments, a machine learning system, model, or neural network described herein is an artificial neural network. In some embodiments, a machine learning system, model, or neural network described herein is a convolutional neural network. In some embodiments, a machine learning system, model, or neural network described herein is a recurrent neural network. In at least one embodiment, a machine learning system, model, or neural network described herein is a Bayes classifier. As used herein, a “machine learning system” may refer to one or multiple machine learning models that cooperatively generate one or more outputs based on corresponding inputs. For example, a machine learning system may refer to any system architecture having multiple discrete machine learning components that consider different kinds of information or inputs.

The method 1244 further includes actuating an actuator of the exercise device based on the determined movement of the simulated object in the simulation video or video information at 1258, and the method 1244 includes moving a contact point of the exercise device relative to the frame of the exercise device with the actuator at 1252. In some embodiments, the frame is further movable relative to the base to change an inclination or declination of the exercise device.

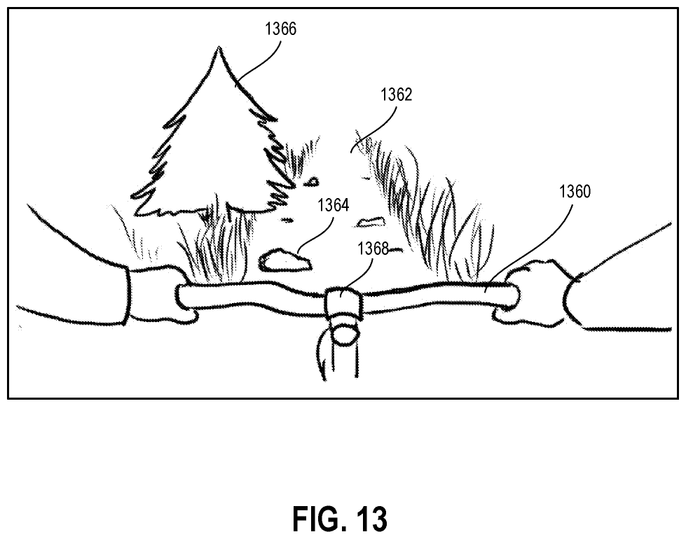

An example of simulated object detection and image processing is illustrated in FIG. 13. The video information of FIG. 13 represents a mountain bike trail ride recorded by the user or another rider. The video information includes the handlebars 1360 (which may be detected as a simulated object for determining haptic information for actuating the handlebars of the exercise device), the trail 1362, trail surface features 1364 (such as rocks and roots), and environmental objects 1366 that may be used as reference points. In some embodiments, the system may detect the handlebars 1360 through image recognition, edge detection, or other image processing techniques to identify the expected shape of either straight handlebars or drop handlebars. Because part or all of the handlebars 1360 may be obscured or out of the video frame during portions of the video information. In some embodiments, the system may identify a stem 1368, which connects the handlebars 1360 to a frame of the bicycle as a proxy for the position and orientation of the handlebars 1360.

In some embodiments, the handlebars 1360 are a simulated object, and the system may determine the haptic information based on the position and movement of the handlebars 1360 relative to one or more reference points, such as the trail 1362, trail surface features 1364, or other environmental objects 1366. In some embodiments, the system may detect more than one simulated object to provide haptic simulation with a plurality of actuators. For example, the handlebars 1360 in the video information may be detected and tracked to calculated haptic information used to actuate a first actuator (or actuators) associated with the handlebars of the exercise device, and trail surface features 1364 may be detected and tracked to approximate the objects interacting with the rear wheel of the bicycle to provide haptic simulation for the saddle movement.

In at least one example, the recorded video information and/or simulation data includes global position system (GPS) information to correlate the video location to a real-world location. The GPS information may be used to determine an elevation profile and approximate the inclination or declination of the frame of the exercise device relative to a base.

In at least one embodiment, a system or method according to the present disclosure allows for a user of an exercise device to be more immersed in a video or audio presentation during a workout routine. In some examples, simulation data includes haptic information that is coordinated to the video information and/or audio information. In some examples, the haptic information may be calculated by object detection or other image processing and recognition techniques to create immersive haptic simulation of conventional action camera videos.

### INDUSTRIAL APPLICABILITY

The present disclosure relates generally to systems and methods for haptic simulations in an exercise device. More particularly, the exercise devices of the present disclosure include one or more contact points of the exercise device that are movable relative to a frame of the exercise device to increase the user's immersion in a simulated environment.

An exercise device is any mechanical device that is used to provide or replicate a physical activity in a localized space. Exercise devices can include a treadmill, cable or spring resistance machine, weight resistance machine, dumbbells, elliptical machine, stepper machine, stationary bicycle, rowing machine, or any other machine or exercise device. In an example, it should be understood that while a road bicycle may not be an exercise device, as used herein, a bicycle positioned on a stationary trainer device should be considered an exercise device as the bicycle remains in one location while the user rides the bicycle on the stationary trainer device.

In some embodiments, an exercise device includes or is in communication with a display. The display allows a user of the exercise device to view video information as the user engages in exercises. In some embodiments, the display presents video information that simulates a route, path, track, road, trail, or other environment associated with the activity replicated by the exercise device. For example, a display integrated in or in communication with a treadmill may present video information simulating traveling down a road in Oahu, Hawaii at a speed approximately equal to the speed at which the tread belt is moving on the treadmill. The resulting experience for the user is a simulated run down the road presented on the display. Similarly, in another example, a display integrated in or in communication with a stationary bicycle may present video information simulating traveling down a mountain trail in Sedona, Arizona at a speed approximately equal to the speed at which the user moves the pedals of the stationary bicycle. The resulting experience for the user is a simulated mountain bike ride down the trail presented on the display. In yet another example, a display integrated in or in communication with a rowing machine may present video information simulating rowing down the Charles River in Cambridge, Massachusetts at a speed approximately equal to the speed at which the user pulls the handle of the rowing machine. The resulting experience for the user is a simulated row down the river presented on the display.

The exercise device simulates the experience based on simulation data that includes video information as described above. In some embodiments, the simulation data includes audio information. For example, the exercise device may offer one or more simulations such as simulate racing in a stage of a bike race or running away from a dinosaur. In such examples, audio information can increase the immersion of a bike race simulation by simulating cheering fans or sound of another racer approaching from behind on a climb. In another example, audio information can increase the immersion of a dinosaur chase by simulating the roar of the dinosaur behind the runner.

In some embodiments, the simulation data includes haptic information. In the previous examples, the exercise device offers simulations that simulate racing in a stage of a bike race or running away from a dinosaur. In such examples, haptic information can increase the immersion of a bike race simulation by simulating rapidly translating and/or rotating the handlebars to simulate the cobblestone road surface of the Paris-Roubaix bicycle race. In another example, haptic information can increase the immersion of the dinosaur chase by translating the tread belt of the treadmill vertically to simulate the ground shaking with the dinosaur footsteps.

The haptic information may be predetermined and stored on a hardware storage device with the video and/or audio information of the simulation data. In some embodiments, the haptic information may be calculated by the exercise device, a client device, or a workout server based on the video information. For example, a recorded video of a mountain bike ride from a rider's viewpoint (such as video information recorded by a GoPro or other action camera) may include the bicycle handlebars within the frame of the video information. In some embodiments, the system detects the location of the handlebars in the video information and determines the movement of the handlebars relative to the trail surface. The movement of the handlebars may be presented to the exercise device as haptic information and allow the exercise device to simulate the movement of the handlebars in the video information by moving the handlebars of the stationary bicycle relative to a frame of the stationary bicycle.

In some embodiments, an exercise device is a stationary bicycle with a display integrated into the exercise device. In some embodiments, the display may be independent from, but in data communication with, the exercise device and receive video information from a computing device of the exercise device. The computing device includes a processor and a hardware storage device with instructions stored thereon that, when executed by the processor, cause the exercise device to perform any of the methods described herein.

In some embodiments, the hardware storage device is any non-transient computer readable medium that may store instructions thereon. The hardware storage device may be any type of solid-state memory; volatile memory, such as static random access memory (SRAM) or dynamic random access memory (DRAM); or non-volatile memory, such as read-only memory (ROM) including programmable ROM (PROM), erasable PROM (ERPOM) or EEPROM; magnetic storage media, such as magnetic tape; platen-based storage device, such as hard disk drives; optical media, such as compact discs (CD), digital video discs (DVD), Blu-ray Discs, or other optical media; removable media such as USB drives; non-removable media such as internal SATA or non-volatile memory express (NVMe) style NAND flash memory, or any other non-transient storage media. In some embodiments, the hardware storage device is local to and/or integrated with the computing device. In some embodiments, the hardware storage device is accessed by the computing device through a network connection.

The exercise device includes one or more contact points supported by a frame with which the user touches, contacts, or engages with the exercise device during the exercise. It should be understood that while the user may touch or contact one or more controls of the exercise device, such as a touchscreen of the display or other input devices to provide inputs to the computing device (e.g., volume controls, resistance levels, power buttons), the controls or input devices are not considered the contact points of the exercise device for the purposes of the exercise performed on the exercise device. In some embodiments, the exercise device is a stationary bicycle, and the intended exercise of the stationary bicycle is cycling, therefor, the components of the stationary bicycle used for cycling are considered to be the contact points of the exercise device. The contact points of the exercise device include the handlebars, the saddle, and the pedals.

The contact points of the exercise device are movable relative to the frame by one or more actuators that are in data communication with the computing device. A first actuator is positioned between the frame and the handlebars to support the handlebars and move the handlebars relative to the frame in response to simulation data processed by the processor of the computing device.

Each of the actuators can include one or more individual actuatable devices, such as including a pair of actuatable devices to allow movement of the contact point in plurality of directions or axes relative to the frame. In some embodiments, the actuators are electromagnetic actuators that use permanent magnets, electromagnets, or combinations thereof to apply magnetic force to move the contact point relative to the frame. In some embodiments, the actuators are fluid piston actuators that use compressible or incompressible fluid in a cylinder to apply a force to a piston that is slidable relative to the cylinder. The fluid may be a gas (e.g., pneumatic piston and cylinder) or a liquid (e.g., hydraulic piston and cylinder) that moves piston relative to the cylinder to move the contact point relative to the frame. In some embodiments, the actuators are mechanical actuators that use gears, springs, elastic or biasing elements, or combinations thereof to apply a compression and/or tension force to move the contact point relative to the frame.

In different embodiments, the actuators move the contact points in different path directions and/or shapes. For example, the actuators can move the contact point(s) in a linear path. The first actuator connected to the handlebars may move the handlebars in a linear path up and down to simulate rough surfaces. The second actuator connected to the saddle moves the saddle in a linear path up and down to simulate rough surfaces.

In some examples, the actuators can move the contact point(s) in an arcuate path. The first actuator connected to the handlebars may move the handlebars in an arcuate path up and toward the user or down and away to simulate rough surfaces. The second actuator connected to the saddle may move the saddle in an arcuate path up and toward the user or down and away to simulate rough surfaces. In some embodiments, the travel paths and/or directions can be different between contact points. For example, the handlebars may have an arcuate path and the saddle may have a linear path.

In some examples, an actuator moves the contact point around a rotational axis. For example, the first actuator may rotate the handlebars around a longitudinal axis to simulate the handlebars tilting as would be expected during riding over uneven terrain and/or under heavy pedal loads. The different travel paths or rotational axes may be combined through one or more actuators for a contact point to create a more immersive simulation. In at least one example, the first actuator that moves the handlebars may allow for both rotational movement and linear movement, such as lifting the handlebars while tilting to the user's left.

The actuators move in response to the simulation data provided by the computing device. The simulation data includes haptic information that instructs one or more actuators to move a contact point coordinated with audio and/or video information of the simulation data that is presented to the user on the display or other audiovisual devices (e.g., speakers or headphones). The haptic information may include instructions for one or more actuators to move the contact point(s) a nominal or relative (to a total range of motion) amount, at a particular speed, for a particular duration, or combination thereof.

For example, the haptic information may instruct the first actuator of the handlebars to move the handlebars in coordination with a tree stump presented on the display. The haptic information may instruct the actuator at a 3:26 timestamp to translate the handlebars upward by 35 millimeters (mm) at a rate of 100 millimeters per second (mm/s) and return the handlebars to the original position at a rate of 50 mm/s. The higher rate of upward travel may replicate the compression of the fork of the bicycle impacting the tree stump in the video information, while the lower rate of downward travel may replicate the freefall of the handlebars 108 dropping off the top of the tree stump.

In another example, the haptic information provides different instructions to the different actuators. For example, the haptic information may instruct the first actuator to move the handlebars by 40 mm while the haptic information instructs the second actuator to move the saddle by 25 mm to simulate different wheel paths or different suspension responses to the objects in the displayed video information.

In some embodiments, a treadmill includes at least one actuator for haptic simulation underneath the tread belt. The actuator is in communication with the computing device of the exercise device to receive instructions to move at least a portion of the tread belt in coordination with audio and/or video information presented on the display.

As described herein, the actuator(s) may be an electromagnetic actuator, a fluid piston actuator, a mechanical actuator, or combinations thereof. In some embodiments, the tread belt is a flexible and/or elastic material and a plurality of actuators move different portions of the tread belt to create contours in the surface of the tread belt to simulate a surface presented on the display. For example, a plurality of actuators may move the surface of the tread belt by different amounts in different places to simulate running on a presented beach surface with rippled sand. In another example, the plurality of actuators may move the surface of the tread belt by different amounts in different places to simulate trail running with rocks or roots in the presented trail surface. In another example, the plurality of actuators may move the surface of the tread belt by different amounts in different places to simulate urban running with curbs or potholes in a presented road.

In some embodiments, the entire surface of the tread belt may be movable by the actuator(s) to simulate a global effect in the presented simulation. For example, the entire surface of the tread belt may displace upward before returning to the original position to simulate an explosion in the video information presented on the display. In at least one example, the exercise device may simulate an action scene and prompt the user of the exercise device to run to safety by evading objects and/or clearing obstacles in the presented video and/or audio information. The action scene may simulate escaping a boobytrapped temple where the user must run along uneven pathways, crumbling walls, and escape a large boulder following the user. The actuator(s) may move portions or all of the tread belt to simulate the debris from the walls on the running surface and simulate the shaking of the ground as the boulder rolls behind the user.

In some embodiments, an exercise device has handlebars that are movable relative to a frame. In some embodiments, the handlebars or other contact point are movable relative to the frame by a single-axis actuator. The single-axis actuator may move the handlebars relative to the frame in response to haptic information included in the simulation data.

In some embodiments, the single-axis actuator is in data communication with the computing device of the exercise device to receive instructions including when to actuate, how much to move, how much force to apply, how fast to move, or combinations thereof. For example, a simulation of riding along a rocky trail may include rapid movements of the handlebars. In another example, a simulation of an undulating singletrack or bike path may include more gradual movements of the handlebars along the same axis.

The range of motion of the actuator and a range of motion of the contact point movable by the actuator may be different. For example, the actuator may be connected to the contact point (e.g., the actuator that moves the handlebars) by being coupled to a linkage or lever that moves the contact point. In other examples, the range of motion of the actuator and a range of motion of the contact point movable by the actuator may be equal, as the actuator and the contact point are directly coupled to one another.

In some embodiments, the range of motion of the contact point is in a range having an upper value, a lower value, or upper and lower values including any of 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, or any values therebetween. In some examples, the range of motion of the contact point is greater than 5 mm. In other examples, the range of motion of the contact point is less than 100 mm. In yet other examples, the range of motion of the contact point is between 5 mm and 100 mm. In yet other examples, the range of motion of the contact point is between 10 mm and 80 mm. In yet other examples, the range of motion of the contact point is about 50 mm.

In some embodiments, the maximum rate of motion of the contact point is in a range having an upper value, a lower value, or upper and lower values including any of 50 mm/s, 100 mm/s, 150 mm/s, 200 mm/s, 250 mm/s, 300 mm/s, 350 mm/s, 400 mm/s, 450 mm/s, 500 mm/s, 550 mm/s, 600 mm/s, 650 mm/s, 700 mm/s, 750 mm/s, 800 mm/s, 850 mm/s, 900 mm/s, 950 mm/s, 1000 mm/s, or any values therebetween. In some examples, the maximum rate of motion of the contact point is greater than 50 mm/s. In other examples, the maximum rate of motion of the contact point is less than 1000 mm/s. In yet other examples, the maximum rate of motion of the contact point is between 50 mm/2 and 1000 mm/s. In yet other examples, the maximum rate of motion of the contact point is between 100 mm/s and 750 mm/s. In yet other examples, the maximum rate of motion of the contact point is greater than 500 mm/s.

In another embodiment, an exercise device includes a handlebar with a plurality of actuators that provide multi-axis movement of the handlebars. In the illustrated embodiment, the actuators are each directly coupled to the handlebars at rotational couplings, respectively.

The first actuator and second actuator may actuate and/or move the handlebar approximately the same, (e.g., same amount of motion, same rate of motion) such that the handlebars travel along an axis parallel to the axis of the actuators. In such an example, the rotational couplings may remain at the same angle relative to the handlebars.

The first actuator may move a different amount or at different velocity (magnitude or direction) than the second actuator. The rotational couplings may allow the handlebars to rotate around an axis perpendicular to the axis of motion of the actuators. In some embodiments, the first actuator and the second actuator may move in the same direction but different amounts to simulate both a displacement and a rotation of the handlebars relative to the frame.

In some embodiments, an exercise device includes an actuator configured to move a saddle relative to a frame of the exercise device. The actuator may move the saddle according to haptic information provided by the computing device.

In some embodiments, the saddle is movable in relation to the frame to simulate movement associated with audio and/or video information presented to the user via a display or other device. The actuator may move the saddle in an axis. In some embodiments, the saddle may be connected to a plurality of actuators to move the saddle in a different path or around a rotational axis to simulate other types of movement according to the haptic information.

Some exercise devices include an actuator configured to move a saddle relative to a frame of the exercise device. The actuator may move the saddle according to haptic information provided by the computing device. In some embodiments, the frame is movable (e.g., tiltable) relative to a base that contacts the ground. The frame may connect to the base at a rotational pivot that allows the frame supporting the saddle, handlebars, and pedals to move relative to the ground. In some examples, the frame tilts around the pivot allows the exercise device to simulate the angle of climbing up a road, while the haptic simulation of the saddle along the axis allows the exercise device to simulate the surface condition of the road. The computing device may be in data communication with the actuator(s) and a motor to tilt the frame to provide a more immersive simulation.

While the movable frame relative to the base is described herein in relation to an exercise bicycle, it should be understood that a tiltable or movable frame with haptic simulation, where the frame is movable relative to a base, is applicable to other types of exercise devices, such as treadmills, rowing machines, and other devices.

While a user may maintain substantially continuous contact with the contact points of an exercise bicycle, a user will continuously lift, move, and lower their feet relative to the contact point (i.e., tread belt) of a treadmill. In some embodiments, an exercise device according to the present disclosure may use contact detection and/or prediction to determine when and/or where to move the contact point(s). In some embodiments, the exercise device may adapt the user's position on the exercise device by moving the contact point at or near the location of a footstrike. In some embodiments, the exercise device may conserve energy by only moving the contact point at or near the location of a footstrike.

In some embodiments, the exercise device detects the location and time of the footstrike. For example, the exercise device may include one or more pressure sensors positioned in or underneath the tread belt to measure an application of force to the tread belt. The computing device in communication with the pressure sensor(s) may receive the measurements from the pressure sensor(s) and determine the user's foot has contacted the tread belt at the location of the pressure sensor(s). In some embodiments, a minimum force or pressure measurement may be required for the pressure sensor to transmit the measurement or for the computing device to interpret the measurement as a footstrike. The computing device may then send instructions to the actuator(s) associated with the location of the footstrike to move the tread belt and provide haptic simulation in coordination with audio and/or video information.

In some embodiments, the exercise device includes one or more cameras positioned and oriented to monitor the movement and location of the user's feet relative to the tread belt. The camera may transmit video data to the computing device to allow the computing device to identify the location of the user's foot when the foot make contact with the tread belt and determine the location of the footstrike. The computing device may then send instructions to the actuator(s) associated with the location of the footstrike to move the tread belt and provide haptic simulation in coordination with audio and/or video information.

In some embodiments, the exercise device predicts the location and time of the footstrike. For example, the exercise device may include one or more pressure sensors positioned in or underneath the tread belt to measure an application of force to the tread belt. The computing device in communication with the pressure sensor(s) may receive the measurements from the pressure sensor(s) and determine the user's foot has contacted the tread belt at the location of the pressure sensor(s). In some embodiments, the computing device may track and average the location and intervals between a sequence of footstrikes. The computing device may continue to calculate a rolling average of the recent cadence and/or location of the footstrikes to predict the location of a next footstrike. The computing device may then send instructions to the actuator(s) associated with the location of the predicted footstrike to move the tread belt and provide haptic simulation in coordination with audio and/or video information.

In some embodiments, the exercise device includes one or more cameras positioned and oriented to monitor the movement and location of the user's feet relative to the tread belt. The camera may transmit video data to the computing device to allow the computing device to identify the location of the user's foot when the foot make contact with the tread belt and determine the location of the footstrike. In some embodiments, the computing device may track and average the location and intervals between a sequence of footstrikes. The computing device may continue to calculate a rolling average of the recent cadence and/or location of the footstrikes to predict the location of a next footstrike. The computing device may then send instructions to the actuator(s) associated with the location of the predicted footstrike to move the tread belt and provide haptic simulation in coordination with audio and/or video information.

In some embodiments, the camera may measure the movement of the user's foot and transmit the location and movement of the user's foot to the computing device. The computing device may track the motion of the user's foot immediately prior to the user's foot contacting the tread belt and predict the location of the footstrike. The computing device may then send instructions to the actuator(s) associated with the location of the predicted footstrike to move the tread belt and provide haptic simulation in coordination with audio and/or video information.

To provide haptic simulation of a running surface, the exercise device may include a plurality of actuators to move the tread belt perpendicular to the conventional movement of the rotating tread belt. In some embodiments, the exercise device includes a frame that supports a plurality of actuators to move the tread belt. As described herein, the actuators may include an electromagnetic actuator, a fluid piston actuator, a mechanical actuator, or combinations thereof.

In some embodiments, the exercise device includes a grid of actuators. The grid may position the plurality of actuators in a plurality of columns and rows. In some embodiments, the grid is a 3×5 grid. In some embodiments, the grid has more or less than 3 columns, and in some embodiments, the grid has more or less than 5 rows.

Because the tread belt moves relative to the frame and the actuators, the location of the user's foot will move in relation to the actuators while in contact with the tread belt. In some embodiments, a plurality of actuators in a column moves simultaneously to simulate the user moving relative to the running surface. In some embodiments, the plurality of actuators in a column moves sequentially based on the speed of the tread belt.

In some embodiments, an exercise device treadmill includes actuators positioned in a plurality of columns, where each column includes one actuator that is substantially the full length of the frame. As the user's foot does not move laterally relative to the direction of the tread belt rotation while the user's foot is in contact with the tread belt, the entire column may be actuated to simulate the user moving relative to the running surface.

An exercise device according to the present disclosure may include a plurality of electronic components in data communication. An electronic system includes a processor and a hardware storage device. In some embodiments, the hardware storage device is any non-transient computer readable medium that may store instructions thereon. The hardware storage device may be any type of solid-state memory; volatile memory, such as static random access memory (SRAM) or dynamic random access memory (DRAM); or non-volatile memory, such as read-only memory (ROM) including programmable ROM (PROM), erasable PROM (ERPOM) or EEPROM; magnetic storage media, such as magnetic tape; platen-based storage device, such as hard disk drives; optical media, such as compact discs (CD), digital video discs (DVD), Blu-ray Discs, or other optical media; removable media such as USB drives; non-removable media such as internal SATA or non-volatile memory express (NVMe) style NAND flash memory, or any other non-transient storage media. In some embodiments, the hardware storage device is local to and/or integrated with the processor. In some embodiments, the hardware storage device is accessed by the processor through a network connection.

The processor is further in communication with a display controller. The processor receives simulation data from the hardware storage device. In some embodiments, the simulation data includes video information and haptic information. The processor transmits video information to the display controller and instructions based on the haptic information to the actuator. The display controller communicates with the display to present the video information to a user, while the actuator moves at least one contact point of the exercise device to provide haptic simulation to the user. The processor may coordinate the presentation of the video information and haptic information to the user such that the user perceives the exercise device moving in coordination with the displayed video. In some embodiments, the processor is further in communication with the one or more sensors and/or cameras to monitor the position and/or movement of the user. The processor may transmit instructions or one or more actuators of a plurality of actuators based on the measurements from the sensors and/or cameras.

A method of providing haptic simulation in an exercise device, according to some embodiments of the present disclosure, includes obtaining simulation data for a workout routine performed on an exercise device. In some embodiments, the simulation data is obtained from local hardware storage device. In some embodiments, the simulation data is obtained from a remote hardware storage device, such as by downloading or streaming the simulation data from a remote server or datacenter. A workout routine is a predetermined set of instructions that may be provided to a user. Following the workout routine can guide, entertain, or encourage the user through one or more exercises to produce workout information. The workout routine may include haptic information, video information, audio information, text information, still images, or combinations thereof to communicate the workout routine to the user.

The method further includes displaying video information of the simulation data on a display. In some embodiments, the method further includes playing audio information of the simulation data. In some embodiments, the simulation data includes both audio information and video information. The audio and/or video information provides visual and/or auditory immersion to which the haptic simulation(s) add further immersion.

The haptic information of the simulation data is used in actuating an actuator of the exercise device, and the method includes moving a contact point of the exercise device relative to the frame of the exercise device with the actuator. In some embodiments, the frame is further movable relative to the base to change an inclination or declination of the exercise device.

While the method includes actuating an actuator of the exercise device based on the simulation data, in some embodiments, the simulation data does not include haptic information, and the processor may calculate haptic information from the simulation data.

In some embodiments, the method includes obtaining simulation data for a workout routine performed on an exercise device, similar to or the same as described above. In some embodiments, the simulation data is obtained from local hardware storage device. In some embodiments, the simulation data is obtained from a remote hardware storage device, such as by downloading or streaming the simulation data from a remote server or datacenter.

The method may further include detecting a simulated object in the simulation video or video information and determining a movement of the simulated object in simulation video or video information. In some embodiments, the simulated object is an object in the simulation video or video information that is analogous to a contact point of the exercise device. For example, the simulation video or video information may be a first-person perspective of a rider riding a mountain bike of a trail. The simulation video or video information may include the handlebars in the frame of the simulation video or video information, and a computing device or processor may detect the presence of the handlebars. In other examples, the simulation video or video information may include a running surface in the frame of the simulation video or video information, allowing the system to detected curbs, rocks, or roots in the video and simulate the running surface with actuators that move the tread belt.

The method further includes displaying video information of the simulation data on a display. In some embodiments, the method further includes playing audio information of the simulation data. In some embodiments, the simulation data includes both audio information and video information. The audio and/or video information provides visual and/or auditory immersion to which the haptic simulation(s) add further immersion.

In some embodiments, the simulated object in the simulation video or video information is detected relative to a horizon line or other reference point to measure the amount of displacement of the simulated object in the video. In some examples, the simulated object is measured relative to other stationary objects in the environment to measure the amount of movement. In some embodiments, the movement of the simulated object is determined by a machine learning (ML) that refines through iterations.

A machine learning model (ML model) according to the present disclosure refers to a computer algorithm or model (e.g., a classification model, a regression model, a language model, an object detection model) that can be tuned (e.g., trained) based on training input to approximate unknown functions. For example, a machine learning model may refer to a neural network or other machine learning algorithm or architecture that learns and approximates complex functions and generate outputs based on a plurality of inputs provided to the machine learning model. In some embodiments, a machine learning system, model, or neural network described herein is an artificial neural network. In some embodiments, a machine learning system, model, or neural network described herein is a convolutional neural network. In some embodiments, a machine learning system, model, or neural network described herein is a recurrent neural network. In at least one embodiment, a machine learning system, model, or neural network described herein is a Bayes classifier. As used herein, a “machine learning system” may refer to one or multiple machine learning models that cooperatively generate one or more outputs based on corresponding inputs. For example, a machine learning system may refer to any system architecture having multiple discrete machine learning components that consider different kinds of information or inputs.

The method further includes actuating an actuator of the exercise device based on the determined movement of the simulated object in the simulation video or video information, and the method includes moving a contact point of the exercise device relative to the frame of the exercise device with the actuator. In some embodiments, the frame is further movable relative to the base to change an inclination or declination of the exercise device.

In an example, the video information represents a mountain bike trail ride recorded by the user or another rider. The video information includes the handlebars (which may be detected as a simulated object for determining haptic information for actuating the handlebars of the exercise device), the trail, trail surface features (such as rocks and roots), and environmental objects that may be used as reference points. In some embodiments, the system may detect the handlebars through image recognition, edge detection, or other image processing techniques to identify the expected shape of either straight handlebars or drop handlebars. Because part or all of the handlebars may be obscured or out of the video frame during portions of the video information. In some embodiments, the system may identify a stem, which connects the handlebars to a frame of the bicycle as a proxy for the position and orientation of the handlebars.

In some embodiments, the handlebars are a simulated object, and the system may determine the haptic information based on the position and movement of the handlebars relative to one or more reference points, such as the trail, trail surface features, or other environmental objects. In some embodiments, the system may detect more than one simulated object to provide haptic simulation with a plurality of actuators. For example, the handlebars in the video information may be detected and tracked to calculated haptic information used to actuate a first actuator (or actuators) associated with the handlebars of the exercise device, and trail surface features may be detected and tracked to approximate the objects interacting with the rear wheel of the bicycle to provide haptic simulation for the saddle movement.

In at least one example, the recorded video information and/or simulation data includes global position system (GPS) information to correlate the video location to a real-world location. The GPS information may be used to determine an elevation profile and approximate the inclination or declination of the frame of the exercise device relative to a base.

In at least one embodiment, a system or method according to the present disclosure allows for a user of an exercise device to be more immersed in a video or audio presentation during a workout routine. In some examples, simulation data includes haptic information that is coordinated to the video information and/or audio information. In some examples, the haptic information may be calculated by object detection or other image processing and recognition techniques to create immersive haptic simulation of conventional action camera videos.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the scope of the present disclosure, and that various changes, substitutions, and alterations may be made to embodiments disclosed herein without departing from the scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

It should be understood that any directions or reference frames in the preceding description are merely relative directions or movements. For example, any references to “front” and “back” or “top” and “bottom” or “left” and “right” are merely descriptive of the relative position or movement of the related elements.

The present disclosure may be embodied in other specific forms without departing from its characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. An haptic simulation exercise device comprising:
a frame;
a contact point supported by the frame, the contact point configured to allow user contact with the exercise device during use of the exercise device; and
an actuator configured to displace the contact point relative to the frame.

2. The exercise device of claim 1, wherein the actuator is connected to the frame and the contact point and positioned between the frame and the contact point.

3. The exercise device of claim 1, wherein the actuator is an electromagnetic actuator.

4. The exercise device of claim 1, wherein the actuator is a fluid piston actuator.

5. The exercise device of claim 1, wherein the actuator is a mechanical actuator.

6. The exercise device of claim 1, wherein the contact point is a tread belt.

7. The exercise device of claim 1, wherein the contact point is a handlebar.

8. The exercise device of claim 1, wherein the contact point is a saddle.

9. The exercise device of claim 1, wherein the actuator is a first actuator; and
further comprising a second actuator connected to the contact point, wherein the second actuator is configured to move the contact point relative to the frame.

10. A method of haptic simulation for an exercise device, the method comprising:
obtaining simulation data for a workout routine performed on an exercise device;
displaying video information of the simulation data on a display;
actuating an actuator of the exercise device based on the simulation data; and
moving a contact point of the exercise device relative to a frame of the exercise device with the actuator.

11. The method of claim 10, wherein the simulation data includes haptic information.

12. The method of claim 10, wherein the display is integrated with the exercise device.

13. The method of claim 10, wherein the simulation data includes surface profile haptic information, and
the actuator moves the contact point to simulate a surface in the video information.

14. The method of claim 10, wherein the simulation data includes global positioning system (GPS) information, and
further comprising tilting the frame based on the GPS information.

15. The method of claim 10 further comprising determining haptic information from the video information of the simulation data.

16. The method of claim 15, wherein determining haptic information includes detecting a simulated object in video information of the simulation data, and
determining a movement of the simulated object in the video information.

17. The method of claim 16, wherein detecting a simulated object includes detecting one or more edges of the simulated object relative to a horizon.

18. The method of claim 16, wherein determining a movement of the simulated object includes measuring a speed of the simulated object toward a viewpoint of the video information.

19. A system for haptic simulation with an exercise device, the system comprising:
a frame;
a contact point supported by the frame, the contact point configured to allow user contact with the exercise device during use of the exercise device; and
an actuator configured to displace the contact point relative to the frame;
a display supported by the frame; and
a computing device in data communication with the actuator and the display.

20. The system of claim 19, wherein the computing device includes:
a processor; and
a hardware storage device having instructions stored thereon that, when executed by the processor, cause the system to:
obtain simulation data for a workout routine performed on an exercise device,
display video information of the simulation data on a display,
actuate an actuator of the exercise device based on the simulation data, and
move a contact point of the exercise device relative to a frame of the exercise device with the actuator.
